(19) United States
(12) Patent Application Publication
(10) Pub. No.: US 2009/0215921 A1
(43) Pub. Date: Aug. 27, 2009

(54) MODIFIED DIMETHYLACRYLATE MONOMER, METHOD FOR PREPARING THE SAME, AND POLYMERIC DENTAL COMPOSITE

(76) Inventors: Chih-Ta Lee, Taoyuan Hsien (TW); Key-Yuan Chang, Taoyuan Hsien (TW)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/392,778

(22) Filed: Feb. 25, 2009

(30) Foreign Application Priority Data
Feb. 26, 2008 (TW) 097106597

(51) Int. Cl. A61K 6/093; C07F 7/08

(52) U.S. Cl. 523/116; 556/445

(57) ABSTRACT

A modified dimethylacrylate monomer is represented by the following formula (I):

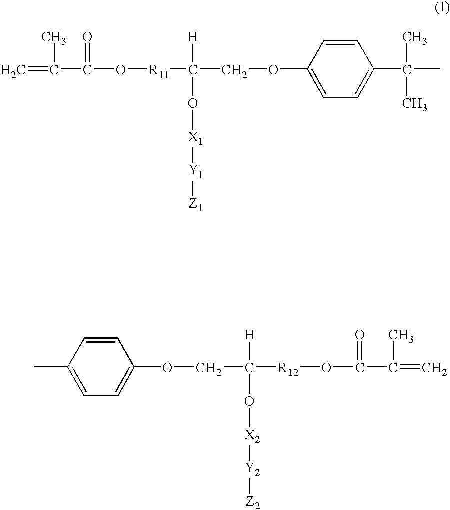

wherein R₁₁ and R₁₂ independently represent a C₁ to C₃ alkylene group or a phenylene group; X₁ and X₂ independently represent NHCO, CO, or a single bond; Y₁ and Y₂ independently represent a C₁-C₁₀ alkylene group or a single bond; and Z₁ and Z₂ independently represent SiA₁A₂A₃ or H, with the proviso that, Z₁ and Z₂ cannot be H at the same time. A method for preparing the modified dimethylacrylate monomer and a polymeric dental composite are also disclosed.

### CROSS-REFERENCE TO RELATED APPLICATION

This application claims priority of Taiwanese Application No. 097106597, filed on Feb. 26, 2008.

### BACKGROUND OF THE INVENTION

1. Field of the Invention

The invention relates to a modified dimethylacrylate monomer, a method for preparing the modified dimethylacrylate monomer, and a polymeric dental composite made from the modified dimethylacrylate monomer.

2. Description of the Related Art

2,2-bis[4-(2-hydroxyl-3-methacryloyloxy)phenyl] propane (Bis-GMA) has been widely used for preparing a dental composite resin because of its superior physical properties, e.g., high strength, after curing.

Since Bis-GMA has a relatively high viscosity, a diluent, e.g., triethylene glycol dimethacrylate (TEGDMA) is usually used in order to reduce the viscosity of the dental restorative. However, addition of the diluent causes a decrease in mole ratio of the Bis-GMA monomer in the dental restorative, thereby resulting in serious polymerization shrinkage after curing. In addition, the two hydroxyl groups of the Bis-GMA molecule are prone to absorb moisture. Thus, a polymerized resin prepared from Bis-GMA is susceptible to swelling by water-absorption so that the bonding force within the polymerized resin is weakened, and inorganic fillers contained in the polymerized resin are likely to be separated from the resin, thereby impairing the properties of the polymerized resin, e.g., inferior strength of adhesion to a tooth, poor abrasion resistance, and decoloring of the resin.

U.S. Pat. No. 7,304,096 discloses an adhesive composition including (a) 1 to 50 wt % of a prepolymer mixture selected from a group consisting of a mixture of 2,2-bis-[4-(2-hydroxy-3-methacryloyloxypropoxy)phenyl]propane (Bis-GMA) of formula (p1) with trifunctional methacrylate (Tri-GMA) of formula (p2), a mixture of Bis-GMA with tetrafunctionalmethacrylate (Tetra-GMA) of formula (p3), and a mixture of Bis-GMA, Tri-GMA and Tetra-GMA; (b) 1 to 30 wt % of an acidic monomer having carboxylic acid or carboxylic anhydride group in a molecule; (c) 1 to 40 wt % of an adhesive monomer; (d) 1 to 10 wt % of a hydrophilic monomer; (e) 0.1 to 5 wt % of an inorganic filler; (f) 10 to 60 wt % of a diluent; (g) 1 to 10 wt % of water; and (h) 1 to 10 wt % of a photoinitiation system, wherein the wt % of all the components are based on the total weight of the composition.

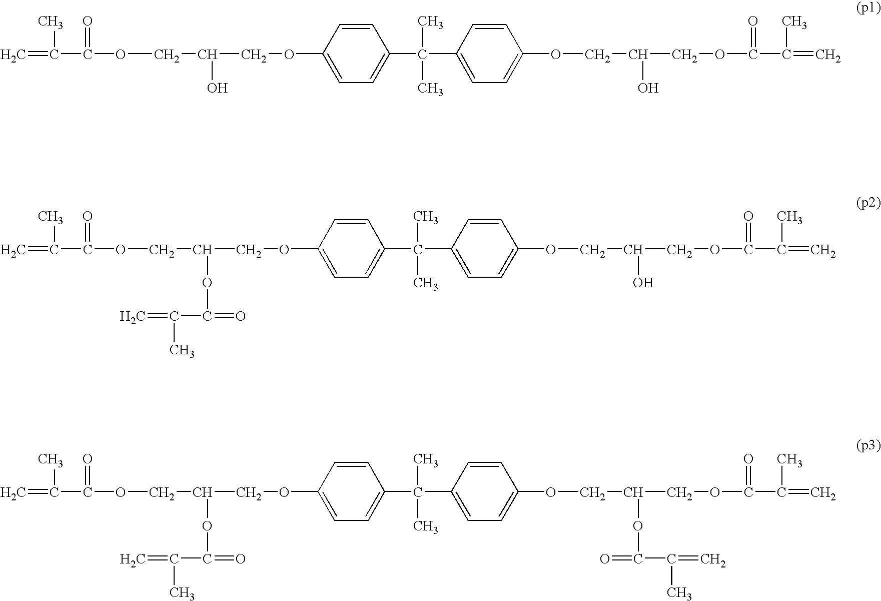

It is noted from Table 3 in this US patent, the adhesive compositions obtained in Examples 1 to 5 have superior polymerization shrinkages (2.2 to 2.7%), but the water absorptions (11 to 14%) thereof remain unsatisfactorily high. Besides, water solubility of each example measures 1.0 to 1.4%, i.e., 1.0 to 1.4% of monomers is not cured upon undergoing curing process and hence is dissolved when the cured adhesive composition is dipped in water. In addition, in U.S. Pat. No. 7,304,096, a diluent, e.g., ethanol or acetone, is still required in the dental adhesive is composition for reducing the viscosity of the composition and for chasing water droplets out of the teeth. Moreover, the composition is used as an adhesive for bonding between the dental composite resin and the teeth, rather than used to act as a dental composite resin by itself.

In Journal of Research of the National Institute of Standards and Technology, 110, 541-558 (2005), Joseph M. Antonucci et al. disclose several organoalkoxysilane compounds derived from Bis-GMA and alkoxy silane compounds. The organoalkoxysilane compounds undergo a complex series of hydrolysis and self-condensation reactions. However, if subject to polymerization, some of the —Si—OH groups which are formed as a result of hydrolysis of the alkoxysilanemoieties in the organoalkoxysilane compounds are left unreacted in subsequent self-condensation reaction, and the presence of residual —Si—OH groups in the resultant polymers will pose similar problems as indicated above, which are attributable to the presence of the hydroxyl groups in the Bis-GMA molecule.

Therefore, there remains a need for a Bis-GMA monomer that exhibits reduced viscosity and water absorption, and is that provides a polymeric dental composite with improved properties, i.e., desirably reduced levels in polymerization shrinkage and water absorption as well as solubility, rather high abrasion resistance and minimized cytotoxicity.

### SUMMARY OF THE INVENTION

According to one aspect of this invention, there is provided a modified dimethylacrylate monomer represented by the following formula (I):

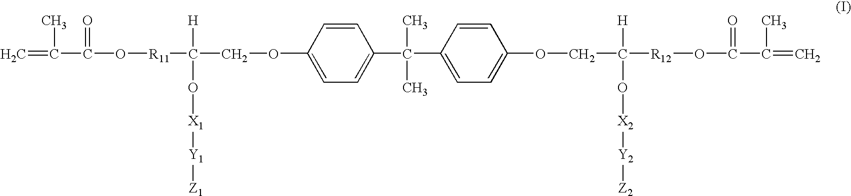

wherein R₁₁ and R₁₂ independently represent a C₁ to C₃ alkylene group or a phenylene group; X₁ and X₂ independently represent NUCO, CO, or a single bond; Y₁ and Y₂ independently represent a C₁-C₁₀ alkylene group or a single bond; and Z₁ and Z₂ independently represent SiA₁A₂A₃ or H, with the proviso that, Z₁ and Z₂ cannot be H at the same time. A₁, A₂, and A₃ independently represent R₂₁B or R₂₁DR₂₂. R₂₁ represents a C₁ to C₁₀ alkylene group or a single bond, B represents NCO, COOH, OH, or H, D represents NHCO, CO, COO, or CHCH, and R₂₂ represents H or a C₁ to C₅ alkyl group which is un-substituted or substituted with a hydroxyl group, with the proviso that, when R₂₂ is H, D cannot be NHCO or CO.

According to another aspect of this invention, a method for preparing the aforesaid modified dimethylacrylate monomer includes reacting a dimethylacrylate monomer represented by the following formula (II):

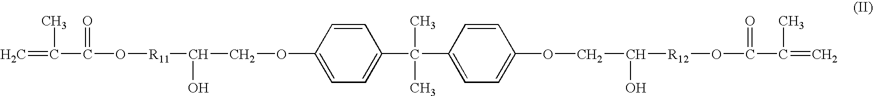

wherein R₁₁ and R₁₂ independently represent C₁ to C₃ alkylene or phenylene,
with a silane compound represented by the following formula (III):

SiA₁A₂A₃A₄ (III)

wherein A₁, A₂, and A₃ independently represent R₂₁B or R₂₁DR₂₂, and A₄ represents R₂₁E, wherein R₂₁ is C₁-C₁₀ alkylene or a single bond, B represents NCO, COOH, OH, or H, D represents NHCO, CO, COO, or CHCH, E represents Cl, Br, NCO, COCl, COOH, OH, or H, and R₂₂ represents H or a C₁ to C₅ alkyl group which is un-substituted or substituted with a hydroxyl group, with the proviso that, when R₂₂ is H, D cannot be NHCO or CO.

According to yet another aspect of this invention, a polymeric dental composite is prepared by reacting a mixture. The mixture contains the aforesaid modified dimethylacrylate monomers, an inorganic filler; and a photo-initiation system.

### DETAILED DESCRIPTION OF THE PREFERRED EMBODIMENTS

A modified dimethylacrylate monomer according to the present invention is shown to include a structure of formula (I):

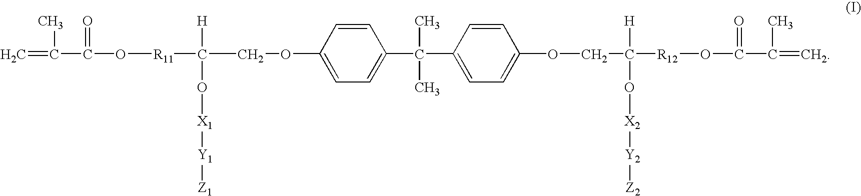

R₁₁ and R₁₂ independently represent a C₁ to C₃ alkylene group or a phenylene group; X₁ and X₂ independently represent NHCO, CO, or a single bond; Y₁ and Y₂ independently represent a C₁-C₁₀ alkylene group or a single bond; and Z₁ and Z₂ independently represent SiA₁A₂A₃ or H, with the proviso that, Z₁ and Z₂ cannot be H at the same time. A₁, A₂, and A₃ independently represent R₂₁B or R₂₁DR₂₂. R₂₁ represents a C₁ to C₁₀ alkylene group or a single bond, B represents NCO, COOH, OH, or H, D represents NHCO, CO, COO, or CHCH, and R₂₂ represents H or a C₁ to C₅ alkyl group which is un-substituted or substituted with a hydroxyl group, with the proviso that, when R₂₂ is H, D cannot be NHCO or CO.

Preferably, in formula (I), R₁₁ and R₁₂ independently represent a methylene group.

Preferably, B represents NCO, COOH, or H.

Preferably, R₂₂ represents H or an un-substituted C₁ to C₅ alkyl group.

Preferably, X₁, X₂, Y₁, and Y₂ are independently a single bond, Z₂ is H, and Z₁ is SiA₁A₂A₃. A₁, A₂, and A₃ are independently R₂₁B. More preferably, R₂₁ is ethylene and B is H.

Preferably, X₁, X₂, Y₁, and Y₂ are independently a single bond, and Z₁ and Z₂ are independently SiA₁A₂A₃. A₁, A₂, and A₃ are independently R₂₁B. More preferably, R₂₁ is ethylene and B is H.

The aforesaid modified dimethylacrylate monomer is prepared by reacting a dimethylacrylate monomer represented by the following formula (II):

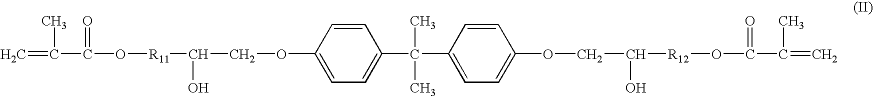

with a silane compound represented by the following formula (III).

SiA₁A₂A₃A₄ (III)

R₁₁ and R₁₂ in formula (II), and A₁, A₂, and A₃ in formula (III) are as defined in formula (I). A₄ represents R₂₁E, wherein R₂₁ is C₁-C₁₀ alkylene or a single bond, and E represents Cl, Br, NCO, COCl, COOH, OH, or H.

Specifically, the dimethylacrylate monomer of formula (II) is dissolved in an organic solvent at ambient temperature followed by slow addition of the silane compound of formula (III) into the organic solvent in an ice bath. The dimethylacrylate monomer of formula (II) and the silane compound of formula (III) are subjected to undergo a substitution reaction in the organic solvent so as to form the aforesaid modified dimethylacrylate monomer of formula (I).

The suitable organic solvent is one that the dimethylacrylate monomer of formula (II) and the modified dimethylacrylate monomer of formula (I) thus obtained can be dissolved therein. Examples of the organic solvent include dichloromethane, trichloromethane, and n-hexane.

Preferably, the substitution reaction is conducted in the presence of an organic base (e.g., triethylamine) having a pH ranging from 8 to 14 or a catalyst (e.g., dibutyltin dilaurate). Since it is well known for a skilled artisan that suitable organic bases or catalysts can be chosen based on the silane compound of formula (III) thus used, further details of the same are omitted herein for the sake of brevity.

The aforesaid modified dimethylacrylate monomers of formula (I) can be used to prepare a polymeric dental composite. To be specific, the polymeric dental composite is prepared by reacting a mixture including the aforesaid modified dimethylacrylate monomers of formula (I), an inorganic filler, and a photo-initiation system.

Preferably, the polymerizing reaction is conducted at a temperature below 60° C.

Preferably, based on the total weight of the mixture, the modified dimethylacrylate monomers of formula (I) are present in an amount ranging from 5 to 60 wt %, more preferably, from 10 to 50 wt %, and most preferably, from 15 to 48 wt %.

Preferably, based on the total weight of the mixture, the inorganic filler is present in an amount ranging from 40 to 95 wt %, more preferably, from 50 to 90 wt %, and most preferably, from 60 to 85 wt %. Suitable examples of the inorganic filler include quartz, silicon, silicon oxide, aluminum oxide, aluminum silicate, barium aluminum silicate, barium sulfate, barium glass, zirconia, or lithium aluminum silicate.

The photo-initiation system includes a photo-initiator and a reductant. Examples of the photo-initiator include, but are not limited to, camphorquinone (CQ), α-diketone aliphatic compound, aromatic carbonyl compound, and tert-amine, which are excited at a wavelength ranging from 400 to 500 nm. Examples of the reductant include, but are not limited to, N,N-dimethylaminoethyl methacrylate (DMAEMA) and ethyl p-dimethyl aminobenzoate (EDMAB). Based on the total weight of the mixture, the photo-initiator is present in an amount ranging from 0.01 to 5 wt %, and the reductant is present in an amount ranging from 0.01 to 5 wt %.

Preferably, the mixture further includes dimethylacrylate monomers of formula (II):

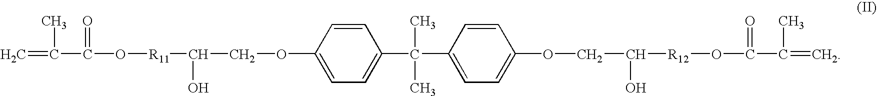

In formula (II), R₁₁ and R₁₂ independently represent a C₁ to C₃ alkylene group or a phenylene group. Preferably, based on the total weight of the mixture, the dimethylacrylate monomers of formula (II) are present in an amount ranging from 5 to 60 wt %, more preferably, from 10 to 50 wt %, and most preferably, from 15 to 40 wt %.

Preferably, the mixture further includes a polymerization inhibitor so as to prevent the mixture from being undesirably subjected to incidental polymerization instorage in the event of exposure to heat or light, thereby prolonging the shelf life of the mixture. Based on the total weight of the mixture, the polymerization inhibitor is present in an amount ranging from 0.01 to 5 wt %. Examples of the polymerization inhibitor include hydroquinone (HQ), hydroquinone monoethyl ether, or hydroquinone monomethyl ether.

Preferably, the mixture further includes a light stabilizer, more preferably, an amine-containing light stabilizer (e.g., Tinubin commercially available from Ciba-Geigy company). Based on the total weight of the mixture, the light stabilizer is present in an amount ranging from 0.01 to 5 wt %.

Preferably, the mixture further includes an anti-oxidant present in an amount ranging from 0.01 to 5 wt % based on the total weight of the mixture. Examples of the anti-oxidant include 2,6-ditert-butyl-4-methyl phenol butylated hydroxytoluene (BHT) and Iganox commercially available from Ciba-Geigy company.

Moreover, the mixture preferably further includes a pigment used for providing color to the polymeric dental composite. The pigment is present in an amount ranging from 0.001 to 0.1 wt % based on the total weight of the mixture.

Preferably, the mixture further includes a diluent. Examples of the diluent include, but are not limited to, ethylene glycol dimethacrylate (EGOMA), diethylene glycol dimethacrylate (DEGDMA), triethylene glycol dimethacrylate, 1,6-bis(methacryloloxy-2-ethoxycarbonylamino)-2,2,4-trimethylhexane, 1,4-butanediol dimethacrylate, 1-methyl-1,3-propanediol dimethyacrylate, and 1,6-hexanediol dimethacrylate. Based on the total weight of the mixture, the amount of the diluent is less than 20 wt %. It should be noted that, since the modified dimethylacrylate monomer of this invention has a relatively low viscosity, the diluent can be dispensed with.

### EXAMPLES

### Sources of Chemicals

1. 2,2-bis[4-(2-hydroxyl-3-methacryloyloxy)phenyl] propane (Bis-GMA): commercially available from Aldrich, CAS no. 1565-94-2.

2. Dichloromethane (CH₂Cl₂): commercially available from ECHO, CAS no. 75-09-2.

3. Triethylamine (C₂H₅)₃N: commercially available from TEDIA, CAS no. 121-44-8.

4. Chlorotriethylsilane: commercially available from TCI, CAS no. 994-30-9.

5. Dibutyltin dilaurate: commercially available from TCI, CAS no. 77-58-7.

6. 3-isocyanatopropyltriethoxysilane (IPTS): commercially available from GE silicones, CAS no. 24801-88-5.

7. Camphorquinone (CQ): commercially available from Aldrich, CAS no. 2767-84-2.

8. Ethyl p-dimethyl aminobenzoate (EDMAB): commercially available from Aldrich, CAS no. 10287-53-3.

9. Hydroquinone (HQ): commercially available from SHOWA, CAS no. 123-31-9.

10. Pigment: Yellow #5 and Yellow #6, commercially available from FD&C.

11. Triethylene glycol dimethacrylate (TEGDMA): commercially available from Aldrich, CAS no. 109-16-0.

### Equipment

1. Evaporator: commercially available from EYELA; model no. NVC-2000.

2. Mixer: commercially available from Labo Plastomill; model 50C150.

3. Nuclear Magnetic Resonance spectrometer (NMR): commercially available from Bruker; model no. ADVANCED 300.

4. Fourier Transform Infrared spectrometer (FT-IR): commercially available from Perkin Elmer; model no. T1.

### General Method

1. Viscosity is measured using a Brookfield viscosmeter.

2. Water absorption for a modified Bis-GMA monomer is measured according to Karl Fischer titration.

3. Polymerization depth for a polymeric dental composite is determined according to ISO-4049. A polymeric dental composite made from the modified dimethylacrylate monomer of this invention (see infra) is filled into a column that is 4 mm in diameter and 10 mm in height and is exposed to a light having a wavelength of 460 nm and an intensity of 1050 mW for 15 seconds. The polymerization depth is then measured.

4. Curing time for a polymeric dental composite is determined according to ISO-4049. The polymeric dental composite is disposed on an aluminum pan of a photo differential scanning calorimeter (PhotoDSC) and is exposed to a light having a wavelength ranging from 400 to 500 nm. The time for curing the polymeric dental composite is then determined.

5. Polymerization shrinkage for a polymeric dental composite is determined according to ISO-4049. The polymeric dental composite having a density (d_{before}) is filled into a cylindrical container and is exposed to a light having a wavelength of 460 nm and an intensity of 1050 mW for 15 seconds so as to obtain a cured composite. The density (d_{after}) of the polymeric dental composite after curing is measured. The polymerization shrinkage is calculated using the following formula:

Polymerization shrinkage (%)=[(1−d_{before})/(1−d_{after})]*100

6. Water absorption and solubility for a polymeric dental composite is determined according to ISO-4049. The polymeric dental composite made from the modified dimethylacrylate monomer of this invention (see infra) is filled into a column that is 10 mm in diameter and 3 mm in thickness and is exposed to a light having a wavelength of 460 nm and an intensity of 1050 mW for 15 seconds so as to obtain a cured test sample. The weight (W₀) of the cured test sample is measured. The cured test sample is dipped into distilled water at 37° C. Every 24 or 48 hours, the test sample is taken out, water is removed from the surface of the sample, and the weight (W₁) of the test sample is measured. The test sample taken out from the water is completely dried in an oven to remove water therein, followed by measuring the weight (W₂) of the dried test sample. The water absorption and solubility are determined using the following formulae:

Water absorption (%) [(W₁−W₀)/W₀]×100

Solubility (%)=[(W₀−W₂)/W₀]×100

7. Compressive strength is measured as follows. A polymeric dental composite made from the modified dimethylacrylate monomer of this invention (see infra) is filled into a column that is 8 mm in diameter and 3 mm in thickness and is exposed to a light having a wavelength of 460 nm and an intensity of 1050 mW for 15 seconds so as to obtain a cured test sample. The compressive strength is determined using an Instron 5566 universal testing machine (Instron Corp., Canton, Mass., USA) at a crosshead rate of 0.5±0.2 mm/sec.

8. Two-body abrasion is measured based on the method disclosed in U.S. Pat. No. 6,573,312. A polymeric dental composite made from the modified dimethylacrylate monomer of this invention (see infra) is filled into a column that is 120 mm in diameter and 2 mm in thickness and is exposed to a light having a wavelength of 460 nm and an intensity of 1050 mW for 15 seconds so as to obtain a cured test sample. The cured test sample is placed into an abrasion tester (commercially available from Cometech Testing Machines Co., Ltd., model no. QC-619T) and travels 10 m on NO. 400 sandpaper under the weight of 250 g. Two body abrasion is evaluated with thickness decrease and weight change before and after abrasion.

9. Surface hardness is measured based on the method disclosed in U.S. Pat. No. 6,573,312. A polymeric dental composite made from the modified dimethylacrylate monomer of this invention (see infra) is disposed between two parallel glasses separated from each other by a 2 mm gap, and is cured by exposing to a light having a wavelength of 460 nm and an intensity of 1050 mW for 15 seconds so as to form a cured plate sample. Vickers hardness is measured under the weight of 100 g for 10 seconds with a minute durometer.

10. Cytotoxicity tests are conducted in accordance with the method described in ISO 10993-5: Biological Evaluation of Medical Devices-Test for in vitro cytotoxicity. A polymeric dental composite made from the modified dimethylacrylate monomer of this invention (see infra) is filled into a column that is 10 mm in diameter and 2 mm in thickness and is cured by exposing to a light having a wavelength of 460 nm and an intensity of 1050 mW for 15 seconds so as to obtain a test sample. L-929 fibroblasts are diluted in minimal essential medium (MEM) containing 10% of fetal bovine serum (FBS) to 1×10⁵ cells/ml, followed by inoculation into a 6-well culture plate, 2 ml per well. Subsequently, the culture is cultivated in an incubator set at a temperature of 37° C. and 5% of CO₂ for 24 hours. Thereafter, the MEM is removed, and 2 ml of agar medium (in the form of liquid) heated to 45° C. is added to each well of the 6-well culture plate. When the temperature of the agar medium drops to room temperature, the agar medium would coagulate, thereby obtaining a cell-containing solid agar medium. Subsequently, the test sample is placed on the cell-containing agar media and is cultivated in an incubator set at 37° C. and 5% of CO₂ for 24 hours. On the back of each well of the culture plate at a position corresponding to the sample, a profile of the sample and a circle concentric with the profile and having a radius greater than that of the profile are drawn. The area within the profile is a sample zone, and the area outside the profile and within the circle is a diffusion zone. Thereafter, the sample is removed from the surface of the agar medium, and the agar medium is stained using a neutral red solution. Subsequently, the number and morphology of the cells in the sample zone and the diffusion zone are observed under an inverted microscope set at 200× magnification. Azone index and a lysis index are calculated for the sample from the number and morphology of the cells in the sample zone and the diffusion zone, and a response index is calculated from the two indices.

### Preparation of Modified Dimethylacrylate Monomer

### Example 1

### Preparation Steps

(1) 40.05 g of Bis-GMA was dissolved in 200 ml of dicholoromethane at ambient temperature, and 70 ml of triethylamine was added thereto, thereby obtaining a first mixture.

(2) The first mixture was placed in an ice bath and filled with nitrogen gas, and 12.25 g of chlorotriethylsilane was gradually added thereto, thereby obtaining a reaction mixture in which a substitution reaction took place.

(3) After 3 hours, the reaction mixture was subjected to thin-layer chromatography (TLC) to determine whether the substitution reaction was complete (i.e., all of Bis-GMA molecules were reacted with the chlorotriethylsilane), in which a solution of n-hexane and ethyl acetate (7:3) was used as an eluent.

(4) The side-product, triethylamine salt having the following formula (e0), was removed from the reaction mixture by suction filtration.

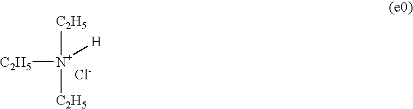

(5) The solvent in the reaction mixture was removed using an evaporator, thereby obtaining a triethyl silane-substituted Bis-GMA having the following formula (e1).

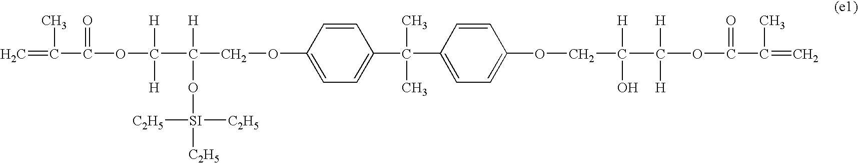

### Structure Identification:

The structure of the triethyl silane-substituted Bis-GMA obtained in Example 1 was identified using NMR and FT-IR. The NMR results are: ¹H-NMR (300 MHz, D-CDCl₃), δ7.12(d, J=8.4 Hz, 4H, Ar), 56.79 (d, J=8.4 Hz, 4H, Ar), 66.11 (br, 2H, methacryl), 55.56 (br, 2H, methacryl), δ 4.30-4.20 (m, 6H, —CH₂ of methacryl, CH), δ3.95-3.87 (m, 4H, CH₂—O—Ar), δ1.94(s, 6H, CH₃ of methacryl), δ1.62(s, 6H, CH₃), 50.92 (t, J=7.7 Hz, 9H, CH₃ of oSiEt), 60.58 (Quartet, J=7.7 Hz, 6H, Si—CH₂—). The IR result shows that the intensity of the OH absorbance peak at 3400 cm⁻¹ was reduced, C═O absorbance peak was observed at about 1721 cm⁻¹, C═C absorbance peak was observed between 1600 cm¹ to 1660 cm⁻¹, and Si—CH₂ absorbance peak was observed between 1150 cm⁻¹ to 1250 cm⁻¹.

### Example 2

The steps for preparing the modified dimethylacrylate monomer according to this invention in Example 2 were substantially the same as those of Example 1. The difference resides in that the amount of the triethylamine was 25 g in Example 2. The modified Bis-GMA thus obtained has the following formula (e2).

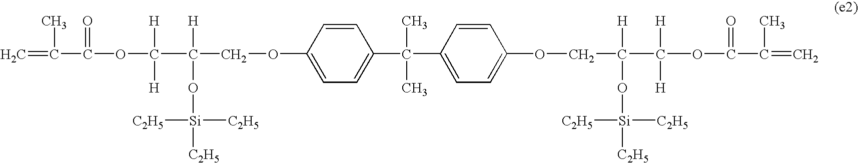

Structure identification:

The structure of the triethyl silane-substituted Bis-GMA obtained in Example 1 was identified using NMR and FT-IR. The NMR results are: ¹H-NMR (300 MHz, D-CDCl₃), δ 7.12(d, J=8.4 Hz, 4H, Ar), 56.79 (d, J=8.4 Hz, 4H, Ar), δ 6.11 (br, 2H, methacryl), 55.56 (br, 2H, methacryl), δ 4. 30-4.20 (m, 6H, —CH₂ of methacryl, CH), δ 3.95-3.87(m, 4H, CH₂—O—Ar), 51.94 (s, 6H, CH₃ of methacryl), 51. 62 (s, 6H, CH₃), 50.92 (t, J=7.7 Hz, 18H, CH₃ of OSiEt), δ 0.58 (Quartet, J=7.7 Hz, 12H, Si—CH₂—). The IR result shows that no intensity was detected for the OH absorbance peak at 3400 cm⁻¹, C═O absorbance peak was observed at about 1721 cm⁻¹, C═C absorbance peak was observed between 1600 cm⁻¹ to 1660 cm⁻¹, and Si—CH₂ absorbance peak was observed between 1150 cm⁻¹ to 1250 cm⁻¹.

### Viscosity and Water Absorption Tests

The modified Bis-GMA obtained in Example 2 and Bis-GMA were subjected to viscosity and water absorption tests. The results are shown in Table 1.

TABLE 1

| | Example 2 | Bis-GMA |
|---|---|---|
| Viscosity (cp) | 440 | 458400 |
| Water absorption | 7.4 | 35 |
| (mg/mm³/week) | | |

It is noted from Table 1 that, compared to un-modified Bis-GMA, the modified dimethylacrylate monomer of formula (e2) has relatively low viscosity and water absorption.

### Experiment

### Preparation of Polymeric Dental Resin Composites

### Experiment 1

26 wt % of Bis-GMA, 20 wt % of the modified Bis-GMA of formula (e1), 45 wt % of SiO₂, 4 wt % of Al₂O₃, 4 wt % of BaSO₄, and 0.2 wt % of HQ were mixed in a mixer. After mixing homogeneously, 0.4 wt % of CQ, 0.2 wt % of EDMAS, and 0.2 wt % of the pigment were added thereto and mixed so as to obtain a polymeric dental composite.

### Experiments 2 to 4

The steps for preparing the polymeric dental composite samples in experiments 2 to 4 were substantially similar to those in experiment 1, except for the amount of Bis-GMA and the amounts and types of the modified Bis-GMA. The amounts of Bis-GMA and the amounts and types of the modified Bis-GMA for experiments 1 to 4 are shown in Table 2.

### Comparative Experiment

26 wt % of Bis-GMA, 20 wt % of TEGDMA, 45 wt % of SiO₂, 4 wt % of Al₂O₃, 4 wt % of BaSO₄, and 0.2 wt % of HQ were mixed in a mixer. After mixing homogeneously, 0.4 wt % of CQ, 0.2 wt % of EDMAB, and 0.2 wt % of the pigment were added thereto and mixed so as to obtain a polymeric dental composite.

TABLE 2

| | | Modified | Modified | |
|---|---|---|---|---|
| | | Bis-GMA of | Bis-GMA of | |
| | Bis-GMA | Example | Example | |
| | (wt %) | 1 (wt %) | 2 (wt %) | TEGDMA |
| Experiment 1 | 26 | 20 | — | — |
| Experiment 2 | 26 | — | 20 | — |
| Experiment 3 | — | 46 | — | — |
| Experiment 4 | — | — | 46 | — |
| Comparative | 26 | — | — | 20 |
| Experiment | | | | |

—: not included

### Property Tests

The polymeric dental composites obtained in experiments 1 to 4 and the comparative experiment were respectively subjected to tests of polymerization depth, is curing time, polymerization shrinkage, water absorption and solubility, compressive strength, two-body abrasion, and surface hardness. The results are shown in Table 3.

### Cytotoxicity Test

Cytotoxicity tests were conducted with respect to the samples obtained in experiments 1 to 4 and the comparative experiment. According to the biological evaluation, a zone index and a lysis index were calculated by observing the number and morphology of cells and with reference to the index definitions in ISO 10993-5. Thereafter, a response index (RI) value was calculated from the two indices using the formula (RI-zone index/lysis index). The lower the RI value, the lower would be the cytotoxicity.

The samples used in the cytotoxicity tests include: (1) samples obtained by curing polymeric dental composites of experiments 1 to 4 and comparative example 1 using the method set forth in the paragraph of Cytotoxicity tests under the subtitle of General Method; (2) a filter paper of the same size which was immersed in 1% of phenol solution and used as a positive control; and (3) a polytetrafluoroethylene (PTFE) sample used as a negative control.

TABLE 3

| | | | | | Comp. |
|---|---|---|---|---|---|
| Property | Exp. 1 | Exp. 2 | Exp. 3 | Exp. 4 | Exp. |
| Polymeri- | 9.45 | 9.43 | 9.10 | 9.12 | 9.15 |
| zation | | | | | |
| depth (mm) | | | | | |
| Curing time | 37.0 | 15.9 | 28.3 | 29.9 | 31.5 |
| (sec) | | | | | |
| Polymeri- | 2.17 | 2.56 | 2.32 | 1.63 | 2.82 |
| zation | | | | | |
| shrinkage | | | | | |
| (%) | | | | | |
| Water | 0.695 | 0.403 | 0.417 | 0.386 | 0.604 |
| absorption | | | | | |
| (%) | | | | | |
| Solubility | 0.0181 | 0.0023 | 0.0107 | 0.0033 | 0.0147 |
| (%) | | | | | |
| Compressive | 340.80 | 346.74 | 317.53 | 343.00 | 266.49 |
| strength | | | | | |
| (MPa) | | | | | |
| Weight loss | 20 | 10 | 25 | 40 | 60 |
| after | | | | | |
| abrasion | | | | | |
| (wt %) | | | | | |
| Thickness | 0.0017 | 0.0013 | 0.0053 | 0.0065 | 0.0069 |
| loss after | | | | | |
| abrasion | | | | | |
| (μm) | | | | | |
| Surface | 17.83 | 18.03 | 16.57 | 18.47 | 17.13 |
| hardness | | | | | |
| (HV) | | | | | |
| Cytotoxicity | 0/0 | 1/1 | 0/0 | 0/0 | 1/1 |
| Test (RI | | | | | |
| value) | | | | | |

It is noted from Table 3, compared to the comparative experiment, the polymeric dental composites of this invention have reduced levels in polymerization shrinkage and water absorption as well as solubility (for Experiments 2 and 3). Moreover, the polymeric dental composites of this invention have superior compressive strength and abrasion resistance, and also passed the cell cytotoxicity test in accordance with ISO 10993-5.

In sum, by virtue of substitution of the hydroxyl group in the Bis-GMA molecule with the specific silicon-containing group, the modified Bis-GMA of this invention has relatively low viscosity and water absorption. Moreover, it is evident that the modified Bis-GMA of the present invention provides the polymeric dental composite formed therefrom with good properties (especially superior compressive strength and abrasion resistance), and is harmless to a human.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A modified dimethylacrylate monomer represented by the following formula (I): 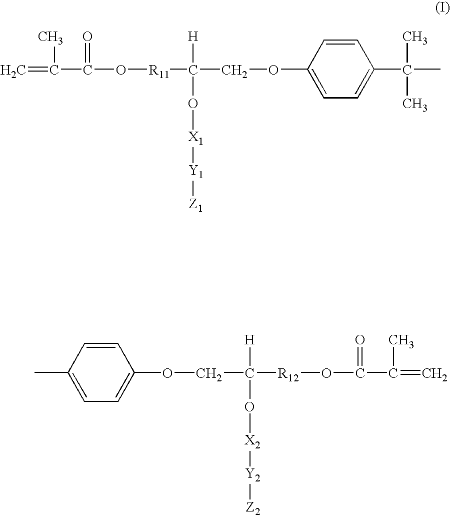
wherein R₁₁ and R₁₂ independently represent a C₁ to C₃ alkylene group or a phenylene group; X₁ and X₂ independently represent NHCO, CO, or a single bond; Y₁ and Y₂ independently represent a C₁-C₁₀ alkylene group or a single bond; and
Z₁ and Z₂ independently represent SiA₁A₂A₃ or H, with the proviso that, Z₁ and Z₂ cannot be H at the same time,
wherein A₁, A₂, and A₃ independently represent R₂₁ B or R₂₁DR₂₂, R₂, represents a C₁ to CIO alkylene group or a single bond, B represents NCO, COOH, OH, or H, D represents NHCO, CO, COO, or CHCH, and R₂₂ represents H or a C₁ to C₅ alkyl group which is un-substituted or substituted with a hydroxyl group, with the proviso that, when R₂₂ is H, D cannot be NHCO or CO.

2. The modified dimethylacrylate monomer of claim 1, wherein R₁₁ and R₁₂ are a methylene group.

3. The modified dimethylacrylate monomer of claim 1, wherein A₁, A₂, and A₃ independently represent R₂₁B or R₂₁DR₂₂, R₂₁ represents a C₁ to C₁₀ alkylene group or a single bond, B represents NCO, COOH, or H, D represents NHCO, CO, COO, or CHCH, and R₂₂ represents H or an un-substituted C₁ to C₅ alkyl group, with the proviso that, when R₂₂ is H, D cannot be NHCO or CO.

4. The modified dimethylacrylate monomer of claim 1, wherein X₁, X₂, Y₁, and Y₂ are independently a single bond; Z₂ is H; and Z₁ is SiA₁A₂A₃, and wherein A₁, A₂, and A₃ are independently R₂₁B.

5. The modified dimethylacrylate monomer of claim 4, wherein R₂₁ is ethylene and B is H.

6. The modified dimethylacrylate monomer of claim 1, wherein X₁, X₂, Y₁, and Y₂ are independently a single bond; Z₁ and Z₂ are independently SiA₁A₂A₃; and A₁, A₂, and A₃ are independently R₂₁B.

7. The modified dimethylacrylate monomer of claim 6, wherein R₂₁ is ethylene and B is H.

8. A method for preparing a modified dimethylacrylate monomer of claim 1, comprising reacting a dimethylacrylate monomer represented by the following formula (II): 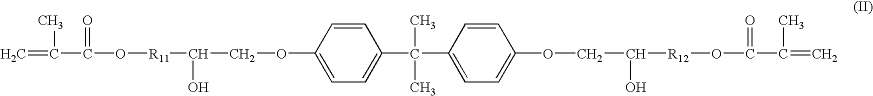
wherein R₁₁ and R₁₂ independently represent C₁ to C₃ alkylene or phenylene,
with a silane compound represented by the following formula (III):
SiA₁A₂A₃A₄ (III)
wherein A₁, A₂, and A₃ independently represent R₂₁B or R₂₁DR₂₂, and A₄ represents R₂₁E, wherein R₂₁ is C₁-C₁₀ alkylene or a single bond, B represents Cl, Br, NCO, COCl, COOH, OH, or H, D represents NHCO, CO, COO, or CHCH, E represents Cl, Br, NCO, COCl, COOH, OH, or H, and R₂₂ represents H or a C₁ to C₅ alkyl group which is un-substituted or substituted with a hydroxyl group, with the proviso that, when R₂₂ is H, D cannot be NHCO or CO.

9. The method of claim 8, wherein the reaction is conducted in the presence of an organic base having a pH ranging from 8 to 14.

10. The method of claim 9, wherein the reaction is conducted in the presence of a catalyst.

11. A polymeric dental composite prepared by reacting a mixture, said mixture containing:
modified dimethylacrylate monomers as claimed in claim 1;
an inorganic filler; and
a photo-initiation system.

12. The polymeric dental composite of claim 11, wherein, based on the total weight of said mixture, said modified dimethylacrylate monomers are present in an amount ranging from 5 to 60 wt %.

13. The polymeric dental composite of claim 11, wherein, based on the total weight of said mixture, said inorganic filler is present in an amount ranging from 40 to 95 wt %.

14. The polymeric dental composite of claim 13, wherein said inorganic filler is selected from the group consisting of; quartz, silicon, silicon oxide, aluminum oxide, aluminum silicate, aluminum barium silicate, barium sulfate, barium glass, zirconium oxide, lithium aluminum silicate, and combinations thereof.

15. The polymeric dental composite of claim 11, wherein said photo-initiation system includes a photo-initiator and a reductant, and wherein, based on the total weight of said mixture, said photo-initiator is present in an amount ranging from 0.01 to 5 wt %, said reductant being present in an amount ranging from 0.01 to 5 wt %.

16. The polymeric dental composite of claim 11, wherein said mixture further includes dimethylacrylate monomers having the following formula (II): 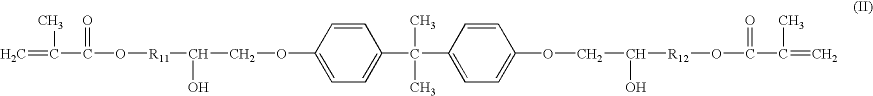
wherein R₁₁ and R₁₂ independently represent a C₁ to C₃ alkylene group or a phenylene group.

17. The polymeric dental composite of claim 16, wherein, based on the total weight of said mixture, said dimethylacrylate monomers having the formula (II) are present in an amount ranging from 5 to 60 wt %.

18. The polymeric dental composite of claim 11, wherein said mixture further includes a polymerization inhibitor having an amount ranging from 0.01 to 5 wt % based on the total weight of said mixture.

19. The polymeric dental composite of claim 18, wherein said polymerization inhibitor is hydroquinone, hydroquinone monoethyl ether, or hydroquinone monomethyl ether.
